(19) United States
(12) Patent Application Publication
(10) Pub. No.: US 2024/0410909 A1
(43) Pub. Date: Dec. 12, 2024

(54) SYSTEM AND METHOD FOR DETECTING CHEMICALLY CONTAMINATED SAMPLES

(71) Applicant: KLA Corporation, Milpitas, CA (US)

(72) Inventors: Mandar Paranjape, San Jose, CA (US); Ozarfar Gafarov, San Jose, CA (US)

(21) Appl. No.: 18/598,906

(22) Filed: Mar. 7, 2024

Related U.S. Application Data
(60) Provisional application No. 63/471,508, filed on Jun. 7, 2023.

(51) Int. Cl. G01N 35/00

(52) U.S. Cl. CPC G01N 35/00613

(57) ABSTRACT

A sample contamination detection assembly may include an environmental sensor device configured to simultaneously measure two or more of humidity, temperature, or volatile organic content of one or more samples to determine chemical contamination. The assembly may include a funnel device. The funnel device may include a plurality of sidewalls that define two or more internal cavities. At least one sidewall may include an inlet opening connected to the inlet cavity and a first exhaust opening connected to the exhaust cavity, where the environmental sensor device may be arranged proximate to the inlet opening and the first exhaust opening. The funnel device may include a lip configured to direct handler air away from the environmental sensor device. The funnel device may include a slit arranged on at least one sidewall, where the slit may be configured to direct air from the one or more samples to the environmental sensor device.

### CROSS-REFERENCE TO RELATED APPLICATION

The present application claims benefit under 35 U.S.C. § 119 (e) of U.S. Provisional Application Ser. No. 63/471,508, filed Jun. 7, 2023, which is incorporated herein by reference in the entirety.

### TECHNICAL FIELD

The present disclosure generally relates to sample fabrication systems and, more particularly, to a system and method for detecting chemically contaminated samples before loading in sample fabrication tools.

### BACKGROUND

Process flows in modern semiconductor device fabrication lines utilize a multitude of fabrication steps performed by a series of semiconductor fabrication tools. For example, metrology tools and inspection tools may be used during the fabrication process to ensure that a certain yield is reached and maintained. Samples in a process flow are typically moved through the process steps in sealable sample transport devices, or front opening unified pods (FOUPs).

Often samples come from the cleaning stage of the sample reclaim process. A majority of the sample reclaim process uses liquid-based chemistry (e.g., through use of water or organic compounds). There is a risk of unintentionally loading a chemically contaminated sample into the inspection tool if one of the process steps is not well controlled (e.g., drying step is not completed properly). As such, the inspection tool may get damaged by unintentionally scanning the chemically contaminated samples. For example, deep ultraviolet (DUV) and extreme ultraviolet (EUV) optics are extremely sensitive to chemical contamination which can alter the performance or cause damage to the tool. Current inspection systems have no prior knowledge of the sample conditions so there is no protection against scanning contaminated samples. Some systems utilize a stainless-steel shield to protect the bottom of the objective lens, however, the shield does not protect the optics from the chemically contaminated sample. Further, the chemically contaminated samples may cause damage to the sample handling system (e.g., robot end effectors, sample chuck, pre-aligners, or the like), and in some instances, adversely affect performance of the vacuum pump and base pressure in vacuum systems.

Therefore, there is a need for a system and method for detecting chemically contaminated samples.

### SUMMARY

A sample contamination detection assembly is disclosed, in accordance with one or more embodiments of the present disclosure. In embodiments, the assembly includes an environmental sensor device, where the environmental sensor device is configured to simultaneously measure at least two or more of humidity, temperature, or volatile organic content of one or more samples to determine chemical contamination of the one or more samples. In embodiments, the assembly includes a funnel device. In embodiments, the funnel device includes a plurality of sidewalls that define two or more internal cavities, where the two or more internal cavities include at least an inlet cavity configured to receive inlet air and an exhaust cavity configured to receive exhaust air, where at least one sidewall of the plurality of sidewalls includes an inlet opening connected to the inlet cavity and a first exhaust opening connected to the exhaust cavity, where the environmental sensor device is arranged proximate to the inlet opening and the first exhaust opening on the at least one sidewall of the funnel device, where at least one additional sidewall of the plurality of sidewalls includes an additional exhaust opening. In embodiments, the funnel device includes a lip configured to direct handler air away from the environmental sensor device. In embodiments, the funnel device includes a slit arranged on at least one sidewall of the plurality of sidewalls, where the slit is connected to the inlet cavity, where the slit is configured to direct air from the one or more samples to the environmental sensor device via the inlet cavity and the inlet opening of the funnel device.

A system for detecting chemical contamination is disclosed, in accordance with one or more embodiments of the present disclosure. In embodiments, the system includes a sample contamination detection assembly. In embodiments, the sample contamination detection assembly includes an environmental sensor device, where the environmental sensor device is configured to simultaneously measure at least two or more of humidity, temperature, or volatile organic content (VOC) of one or more samples. In embodiments, the sample contamination detection assembly includes a funnel device. In embodiments, the funnel device includes a plurality of sidewalls that define two or more internal cavities, where the two or more internal cavities include at least an inlet cavity configured to receive inlet air and an exhaust cavity configured to receive exhaust air, where at least one sidewall of the plurality of sidewalls includes an inlet opening connected to the inlet cavity and a first exhaust opening connected to the exhaust cavity, where the environmental sensor device is arranged proximate to the inlet opening and the exhaust opening on the at least one sidewall of the funnel device, where at least one additional sidewall of the plurality of sidewalls includes an additional exhaust opening. In embodiments, the funnel device includes a lip configured to direct handler air away from the environmental sensor device. In embodiments, the funnel device includes a slit arranged on at least one sidewall of the plurality of sidewalls, where the slit is connected to the inlet cavity, where the slit is configured to direct air from the one or more samples to the environmental sensor device via the inlet cavity and the inlet opening of the funnel device. In embodiments, the system includes a controller communicatively coupled to the sample contamination detection assembly. In embodiments, the controller is configured to execute program instructions causing the one or more processors to: receive one or more reference signals from one or more reference sensor devices; receive one or more sample contamination signals from the environmental sensor device, where the one or more sample contamination signals include the simultaneously measured at least two or more of humidity, temperature, or volatile organic content of the one or more samples; determine chemical contamination of the one or more samples by comparing the received one or more references signals, the one or more sample contamination signals, and one or more predetermined thresholds; and upon determination of chemical contamination of the one or more samples, prevent the one or more samples from being loaded into an inspection chamber of an inspection tool.

A system is disclosed, in accordance with one or more embodiments of the present disclosure. In embodiments, the system a load port device, where the load port device is configured to receive one or more samples from a portion of a sample transport device. In embodiments, the system includes an automated handling sub-system, where the automated handling sub-system is configured to extract the one or more samples from the sample transport device. In embodiments, the system includes a sample contamination detection assembly, where the sample contamination detection assembly is arranged proximate to a load port door of the load port device. In embodiments, the sample contamination detection assembly includes an environmental sensor device, where the environmental sensor device is configured to simultaneously measure at least two or more of humidity, temperature, or volatile organic content of the one or more samples to determine chemical contamination of the one or more samples. In embodiments, the sample contamination detection assembly includes a funnel device. In embodiments, the funnel device includes a plurality of sidewalls that define two or more internal cavities, where the two or more internal cavities include at least an inlet cavity configured to receive inlet air and an exhaust cavity configured to receive exhaust air, where at least one sidewall of the plurality of sidewalls includes an inlet opening connected to the inlet cavity and a first exhaust opening connected to the exhaust cavity, where the environmental sensor device is arranged proximate to the inlet opening and the exhaust opening on the at least one sidewall of the funnel device, where at least one additional sidewall of the plurality of sidewalls includes an additional exhaust opening. In embodiments, the funnel device includes a lip configured to direct air from the automated handling sub-system away from the environmental sensor device. In embodiments, the funnel device includes a slit arranged on at least one sidewall of the plurality of sidewalls, where the slit is connected to the inlet cavity, where the slit is configured to direct air from the one or more samples to the environmental sensor device via the inlet cavity and the inlet opening of the funnel device.

A method is disclosed, in accordance with one or more embodiments of the present disclosure. In embodiments, the method includes opening a lid of a sample transport device using a load port door. In embodiments, the method includes receiving one or more reference signals from one or more reference sensor devices. In embodiments, the method includes receiving one or more sample contamination signals from the environmental sensor device, where the environmental sensor is configured to simultaneously measure at least two or more of humidity, temperature, or volatile organic content of one or more samples. In embodiments, the method includes determining chemical contamination of the one or more samples by comparing the received one or more references signals, the one or more sample contamination signals, and one or more predetermined thresholds. In embodiments, the method includes, upon determination of chemical contamination of the one or more samples, preventing the one or more samples from being loaded into an inspection chamber of an inspection tool.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF THE DRAWINGS

The numerous advantages of the disclosure may be better understood by those skilled in the art by reference to the accompanying figures.

FIG. 1 is a simplified block diagram of a system for detecting chemically contaminated samples, in accordance with one or more embodiments of the present disclosure.

FIG. 2A is a schematic of a funnel device, in accordance with one or more embodiments of the present disclosure.

FIG. 2B is a cross-section view of the funnel device, in accordance with one or more embodiments of the present disclosure.

FIG. 3A is a front perspective view of a sample contamination detection assembly, in accordance with one or more embodiments of the present disclosure.

FIG. 3B is a bottom perspective view of the sample contamination detection assembly, in accordance with one or more embodiments of the present disclosure.

FIG. 3C is a top perspective view of the sample contamination detection assembly, in accordance with one or more embodiments of the present disclosure.

FIG. 3D is a simplified side view of the sample contamination detection assembly, in accordance with one or more embodiments of the present disclosure.

FIG. 3E is a simplified front view of the sample contamination detection assembly, in accordance with one or more embodiments of the present disclosure.

FIG. 3F is a simplified side view of the sample contamination detection assembly, in accordance with one or more embodiments of the present disclosure.

FIG. 4A is a perspective view of a sample device system, in accordance with one or more embodiments of the present disclosure.

FIG. 4B is an exploded view of the sample device system, in accordance with one or more embodiments of the present disclosure.

FIG. 5 is a flowchart depicting a method of detecting chemically contaminated samples, in accordance with one or more embodiments of the present disclosure.

FIG. 6 is a plot depicting humidity and volatile organic content signals over time, in accordance with one or more embodiments of the present disclosure.

FIG. 7 is a plot depicting humidity signals over time as the sample transport device door is open and closed, in accordance with one or more embodiments of the present disclosure.

FIG. 8A is a plot depicting humidity and volatile organic content signals over time for water and isopropyl alcohol, in accordance with one or more embodiments of the present disclosure.

FIG. 8B is a plot depicting humidity and volatile organic content signals over time for water and acetone, in accordance with one or more embodiments of the present disclosure.

### DETAILED DESCRIPTION

The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure. Reference will now be made in detail to the subject matter disclosed, which is illustrated in the accompanying drawings.

Embodiments of the present disclosure are directed to a system and method for detecting chemically contaminated samples. For example, the system and method may include a sample detection assembly configured to detect chemical contamination before inserting the sample, such as a semiconductor wafer, into an inspection chamber to prevent damage to the inspection system. For instance, the sample detection assembly may include, but is not limited to, an environmental sensor device and a funnel device. The funnel device may direct air from the sample transport device (e.g., front opening unified pod (FOUP)) to the environmental sensor while also directing air from a handler away from the environmental sensor, such the environmental sensor detects an accurate signal.

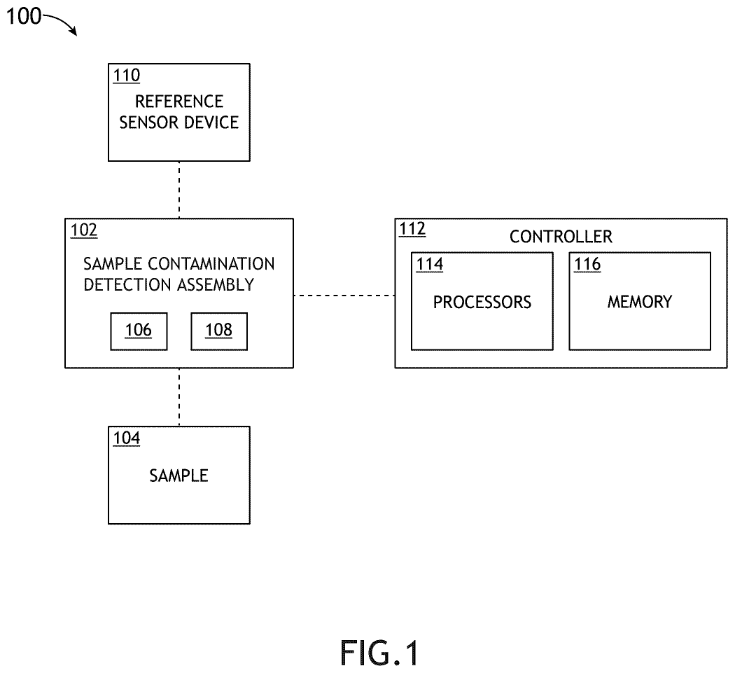

FIG. 1 is a simplified block diagram of a system 100 for detecting chemically contaminated samples, in accordance with one or more embodiments of the present disclosure.

In embodiments, the system 100 includes a sample contamination detection assembly 102 configured to detect chemical contamination of a sample 104. The sample contamination assembly 102 may include, but is not limited to, an environmental sensor device 106 and a funnel device 108. For example, the environmental sensor device 106 may be configured to detect at least one of humidity, temperature, volatile organic content (VOC), or particles. For instance, in a non-limiting example, the environmental sensor device 106 may be configured to simultaneously detect humidity, temperature, volatile organic content (VOC), and particles. By way of another example, the funnel device 108 may be configured to direct air from a sample transport device (e.g., front opening unified pod (FOUP)) to the environmental sensor device 106 while also directing air from a handler away from the environmental sensor device 106. In this regard, the environmental sensor device 106 is able to obtain an accurate contamination signal from the sample 104.

For purposes of the present disclosure, “chemical contamination” may include contamination via a chemical such as, but not limited to, water, organic compounds (e.g., isopropyl alcohol, acetone, or the like), or the like. Further, it is contemplated herein that “chemically contaminated sample” and “wet sample” may be considered equivalent, unless otherwise noted herein. Although embodiments of the present disclosure refer to “chemical contamination” it is contemplated herein that the system and method of the present disclosure may be also used to detect particle contamination to protection inspection systems from particulate contamination eliminating the required cleaning and purge time of the respective inspection systems.

In embodiments, the system 100 includes a reference sensor device 110 configured to obtain reference data. For example, the reference sensor device 110 may include an ambient humidity sensor configured to measure ambient humidity and VOC levels.

In embodiments, the system 100 further includes a controller 112 communicatively coupled to the sample contamination detection assembly 102. The controller 112 may include one or more processors 114 configured to execute program instructions maintained on a memory medium 116. In this regard, the one or more processors 114 of controller 112 may execute any of the various process steps described throughout the present disclosure. For example, the one or more processors 114 of the controller 112 may be configured to determine whether a sample 104 has been chemically contaminated based on test sample data from the environmental sensor device 106 and reference data from the reference sensor device 110. For instance, as will be discussed further herein, the one or more processors 114 of the controller 112 may be configured to compare the test sample data, the reference data, and one or more predetermined thresholds to determine whether the sample 104 has been chemically contaminated. Upon detection of a chemically contaminated sample, the one or more processors 114 of the controller 112 may perform one or more actions. For example, the one or more processors 114 of the controller 112 may alert a user of the presence of the chemically contaminated sample. In one non-limiting instance, inspection of the sample 104 may be paused until the sample 104 is dry. In another non-limiting instance, the chemically contaminated sample 104 may be rejected.

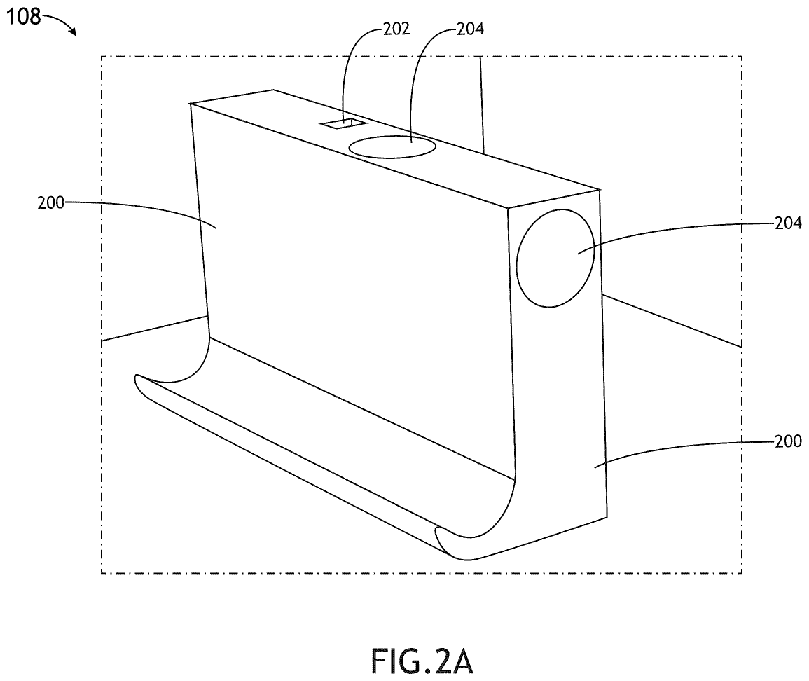

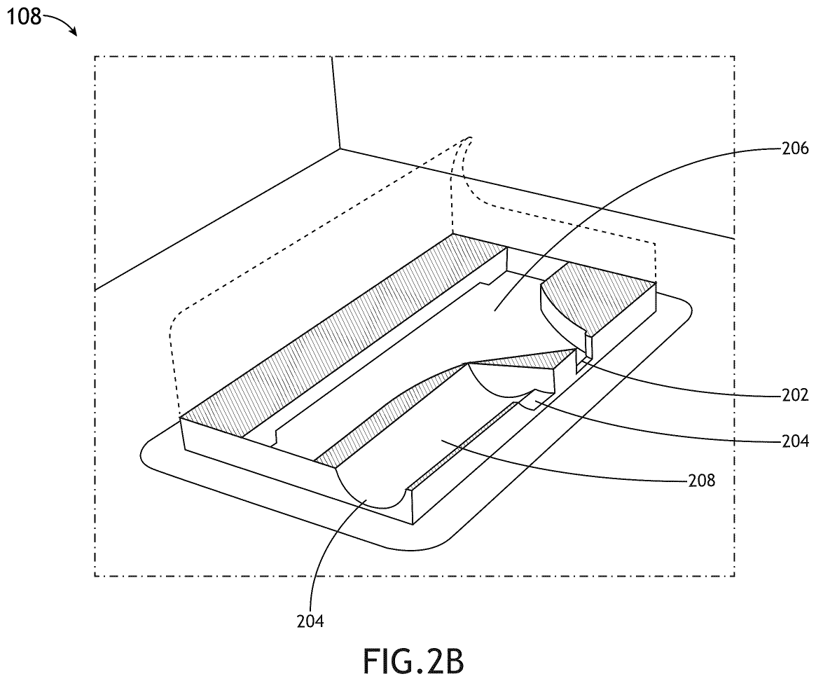

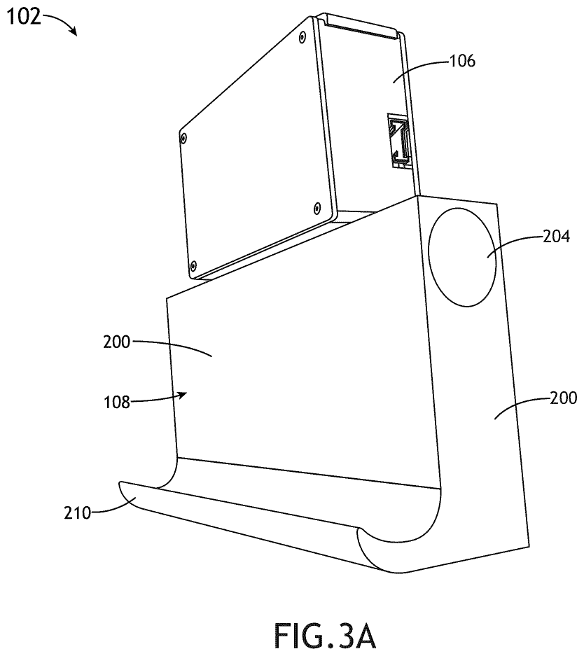

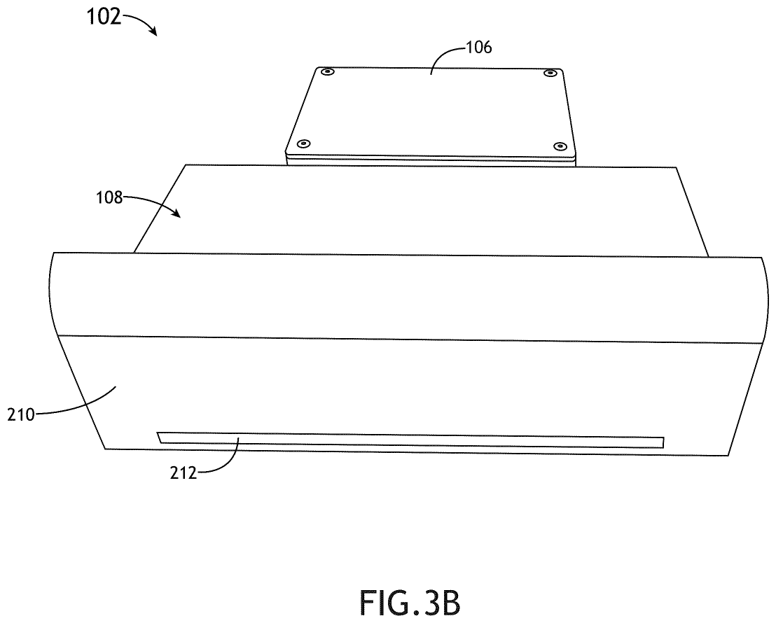

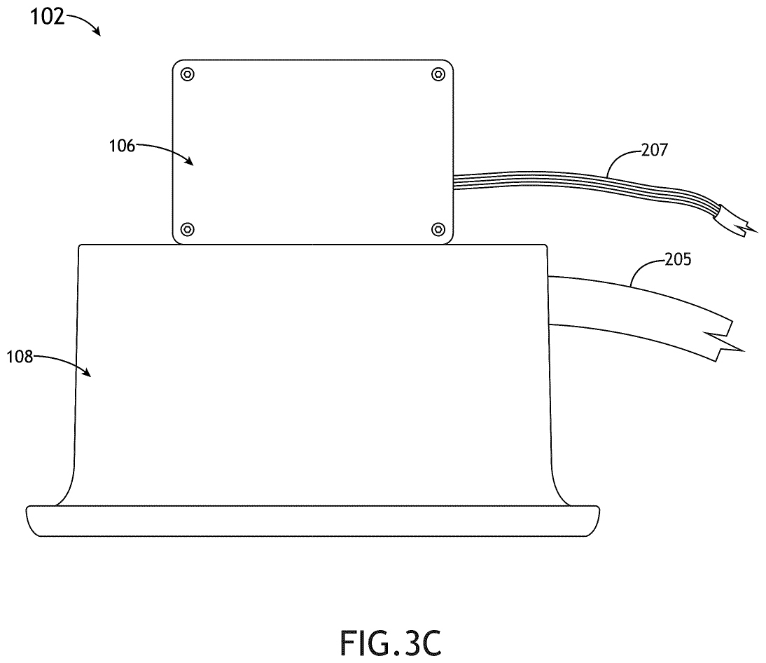

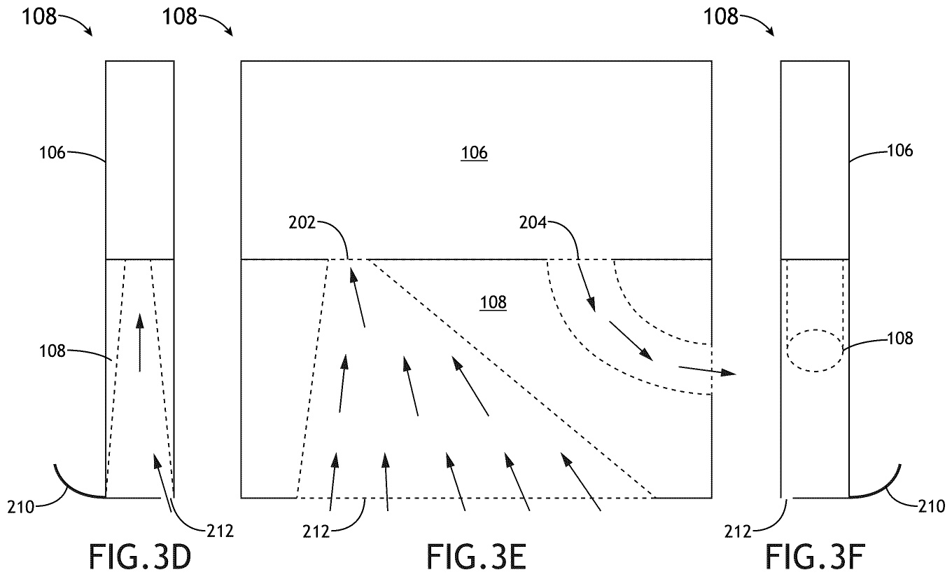

FIGS. 2A-2B are simplified schematics of the funnel device 108 of the sample contamination assembly 102, in accordance with one or more embodiments of the present disclosure. FIGS. 3A-3F are simplified schematics of the sample contamination assembly 102, in accordance with one or more embodiments of the present disclosure.

In embodiments, the funnel device 108 includes a plurality of sidewalls 200 that define one or more internal cavities. For example, the funnel device 108 may include at least a front sidewall, a rear sidewall, a first side sidewall (e.g., left-side sidewall), a second side sidewall (e.g., a right-side sidewall), a top sidewall, and a rear sidewall.

In embodiments, the funnel device 108 includes an inlet opening 202 and one or more exhaust openings 204. For example, as shown in FIGS. 2A-2B, the top sidewall 200 may include the inlet opening 202 and a first exhaust opening 204. Further, at least one of the first side sidewall or the second side sidewall may include a second exhaust opening 204 configured to couple to an exhaust tube 205 (as shown in FIG. 3C). For example, as shown in FIGS. 2A-2B, the second side sidewall may include the second exhaust opening 204 configured to couple to the exhaust tube 205.

In embodiments, the internal cavities defined by the plurality of sidewalls 200 include at least an inlet cavity 206 connected to the inlet opening 202 and an exhaust cavity 208 connected to the one or more exhaust openings. For example, as shown in FIG. 2B the inlet cavity 206 may be configured to receive inlet air from the sample 104 and direct the air to the environmental sensor device 106 through the inlet opening 202. By way of another example, as shown in FIG. 2B, the exhaust cavity 208 may be configured to receive exhaust air from the environmental sensor device 106 through the first exhaust opening 204 in the top sidewall and discard the exhaust air through the second exhaust opening via the exhaust tube 205. For instance, the environmental sensor device 106 may include a fan configured to direct the exhaust air through the first exhaust opening in the top sidewall. In this regard, the exhaust air is directed away from the sample 104 to avoid contamination of the sample 104 (e.g., through debris, particles, or the like).

In embodiments, the environmental sensor device 106 is arranged proximate to the funnel device 108. For example, as shown in FIGS. 3A-3F, the environmental sensor device 106 may be arranged proximate to the inlet opening 202 and the first exhaust opening 204 in the top sidewall of the funnel device 108. In embodiments, the environmental sensor device 106 is coupled to the funnel device 106. For example, the environmental sensor device 106 may be coupled to the top sidewall of the funnel device 108 via any suitable coupling mechanism such as, but not limited to, one or more adhesives, one or more mechanical fasteners (e.g., screws, bolts, or the like), or the like).

In embodiments, the funnel device 108 includes a lip 210. For example, the front sidewall and the bottom sidewall may define the lip 210. For instance, the lip 210 may be shaped as a “J” shape when viewed from the side and include a rounded protrusion. The lip 210 may be configured to direct air from a handler away from the environmental sensor device 106, such the environmental sensor device 106 detects an accurate signal. It is contemplated herein the configuration/shape of the lip 210 shown in FIGS. 2A-3F may be any shape suitable for directing air from the handler away from the environmental sensor device 106.

In embodiments, the funnel device 108 includes a slit 212. For example, as shown in FIG. 3B, the funnel device 108 may include a slit 212 on the bottom sidewall of the funnel device 108. For instance, as shown in FIGS. 3D-3F, the slit 212 may be an additional inlet opening configured to direct air from the sample 104 to the environmental sensor device 106 via the inlet cavity 206 and inlet opening 202.

In embodiments, the environmental sensor device 106 is communicatively coupled to a controller 112. For example, as shown in FIG. 3C, the environmental sensor device 106 may couple to a wire/cable 207, such that the wire/cable 207 is configured to couple the environmental sensor device 106 to the controller 112. Although FIG. 3C depicts a wired connection, it is contemplated herein that the environmental sensor device 106 may be configured to couple to the controller 112 via a wireless connection.

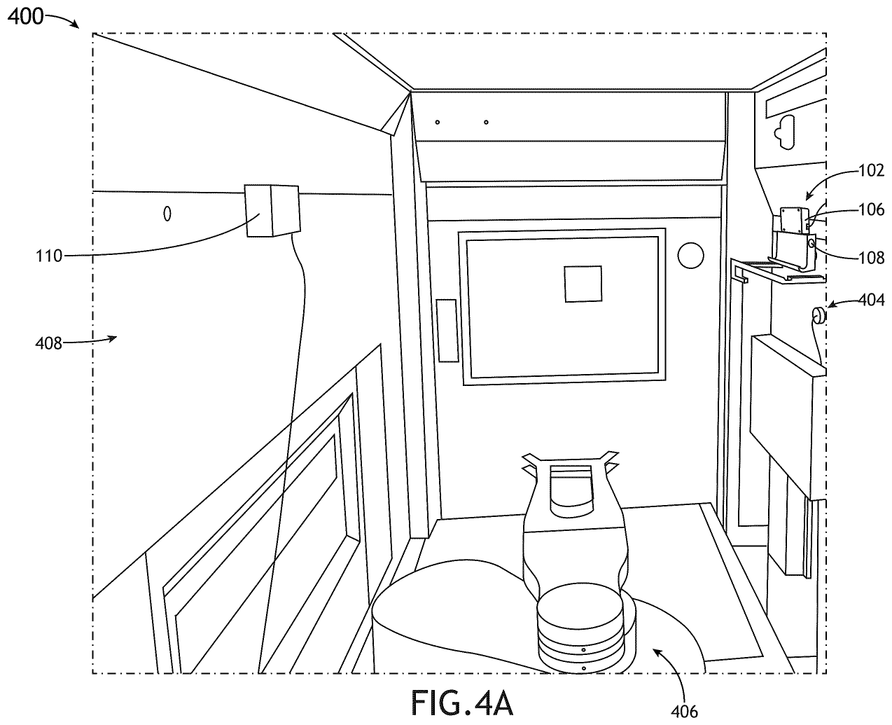

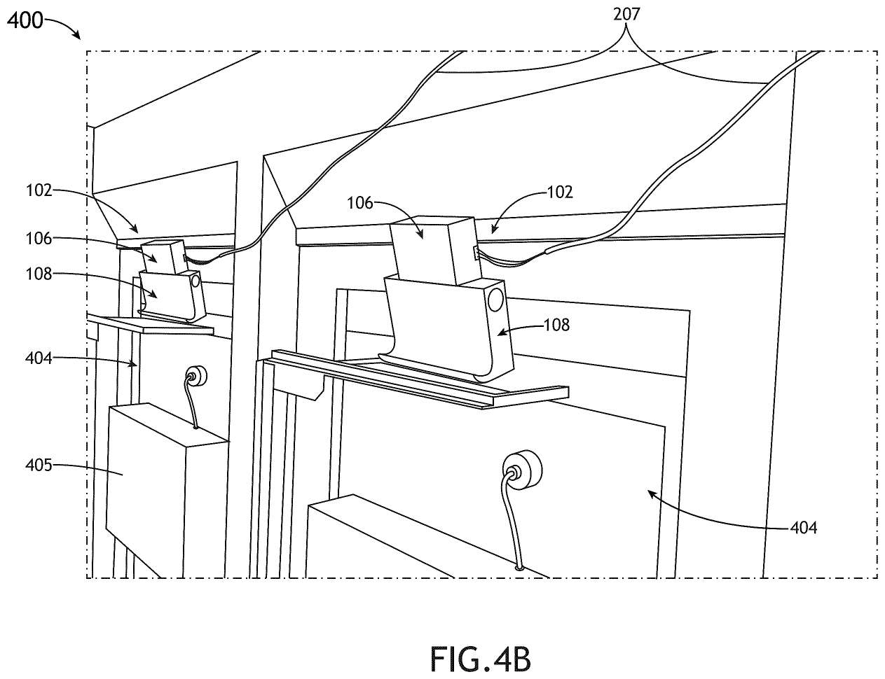

FIGS. 4A-4B illustrate a sample device system 400 integrated with the sample contamination detection assembly 102, in accordance with one or more embodiments of the present disclosure. It is noted that the description of the various embodiments, components, and operations described previously herein with respect to the sample contamination detection assembly 102 should be interpreted to extend to the system 400, and vice versa. Further, it is noted that the description of the various embodiments, components, and operations described previously herein with respect to the sample contamination detection assembly 102 should be interpreted to extend to the system 400, and vice versa.

In embodiments, the system 400 includes a sample transport device (e.g., a FOUP), a load port 404, an automated handling sub-system 406, an inspection chamber 408, and the sample contamination detection assembly 102.

In embodiments, each sample contamination detection assembly 102 is positioned adjacent to an opening of each load port 404. For example, as shown in FIG. 4B, the sample contamination detection assembly 102 is positioned adjacent to a load port door 405 of a first load port 404. In this regard, the slit 212 of the funnel device 108 is arranged proximate to the load port door 405, such that the slit 212 is able to direct air from the sample 104 within the FOUP to the environmental sensor device 106 to detect chemical contamination.

In embodiments, the reference sensor device 110 may be coupled to one or more components of the system 400 to measure a reference signal. For example, in a non-limiting example as shown in FIG. 4A, the reference sensor device 110 may be coupled to a wall of the inspection chamber 408. For instance, the reference sensor device 110 may be arranged on the wall of the inspection chamber 408 a select distance from a sample shutter of the inspection chamber 408. It is contemplated herein that the reference sensor device 110 may be coupled to any component of the system 400 and thus, FIG. 4A should not be construed as limiting the scope of the present disclosure.

In embodiments, the sample contamination detection assembly 102 may be positioned adjacent to the automated handling sub-system 406. For example, the sample contamination detection assembly 102 may be positioned between the load port 404 and the automated handling sub-system 406. In this regard, the sample contamination detection assembly 102 is configured to detect chemical contamination of the sample 104 within the FOUP before allowing the automated handling sub-system 406 to extract the chemically contaminated sample 104 the FOUP.

In embodiments, the FOUP includes a chamber for the one or more samples 104. The FOUP may thus provide a protective environment for the transport of the samples 104 during one or more process steps. The FOUP may further provide a clean environment in which the atmosphere and particulates are controlled.

In embodiments, the FOUP includes one or more storage features suitable for securing items such as, but not limited to, samples 104. The FOUP may incorporate any type of storage features known in the art such as, but not limited to, slots, racks, or fins. Each section of the FOUP (e.g., upper, middle, lower, or the like) may include a plurality of storage features (e.g., slots) within the FOUP to secure one or more samples 104 within the FOUP. In this regard, each section may correspond to a predetermined amount of samples within the FOUP. For example, for 25 samples, the upper section may correspond to samples 1-8, the middle section may correspond to samples 9-16, and the lower section may correspond to samples 17-25.

In embodiments, the FOUP includes one or more components for coupling to additional components such as, but not limited to, an automated handling system, a load port of a tool, or a load port of a buffer station.

In embodiments, the FOUP includes a FOUP lid configured to create a closed, sealed chamber for the samples 104. In embodiments, the load port 404 may be configured to open the lid of the FOUP. For example, the load port 404 may include a load port door 405 configured to open the lid of the FOUP when the door is in close contact with the lid.

In embodiments, the load port 404 may include a purge device configured to blow a predetermined amount of gas (e.g., nitrogen gas) into the FOUP chamber using a purge nozzle. For example, the purge device may be configured to continuously blow a predetermined amount of gas into the FOUP when the load port door 405 is open to prevent the samples from being exposed to ambient air when the load port door 405 is open.

Sample transport devices are generally discussed in U.S. Pat. No. 11,056,366, issued on Jul. 6, 2021, and U.S. Patent Application No. 2021/032776, published on Oct. 21, 2021, which are both incorporated herein by reference in the entirety.

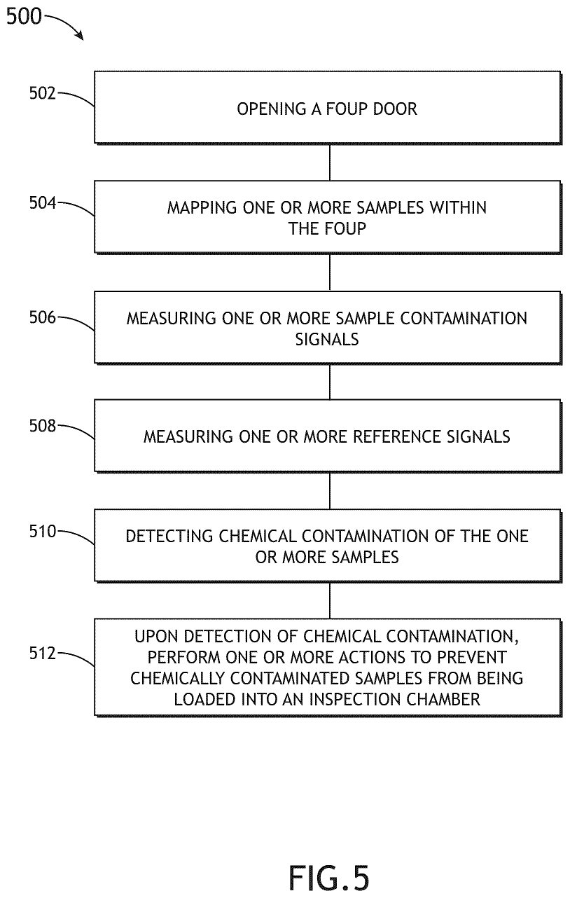

FIG. 5 is a flowchart depicting a method 500 for detecting chemically contaminated samples, in accordance with one or more embodiments of the present disclosure.

In a step 502, a FOUP door may be opened. For example, the load port 404 may include a load port door 405 configured to open the lid of the FOUP when the door is in close contact with the lid. For instance, the load port door 405 may be configured to open the lid by grabbing the lid and translating the lid.

In a step 504, a sample mapper may map the one or more samples within the FOUP and generate sample position data. For example, the sample mapper may provide to generated sample position data to a controller 112.

In a step 506, one or more sample contamination signals may be measured. For example, the environmental sensor device 106 may be configured to detect at least one of humidity, VOC, or particulates. For instance, the slit 212 of the funnel device 108 may direct air from the sample 104 to the environmental sensor device 106.

In a step 508, one or more reference signals may be measured. For example, the reference sensor device 110 may be configured to measure at least one of humidity, VOC, or particulates reference signals. For instance, the reference sensor device 110 may include an ambient humidity sensor configured to measure ambient humidity.

In a step 510, the one or more sample contamination signals (measured in step 506) may be compared to the one or more reference signals (measured in step 508) to determine whether the sample 104 is chemically contaminated. For example, the one or more processors 114 of the controller 112 may be configured to compare the sample contamination signals, reference signals, and one or more predetermined thresholds to determine whether the sample 104 is chemically contaminated. The one or more predetermined thresholds may include one or more user defined thresholds. For example, in a non-limiting example, a threshold indicating contamination may be between approximately 5-10%. As such, if the contamination signals are above the threshold (e.g., 5-10%), the processors 114 of the controller 112 may determine that the sample 104 is chemically contaminated.

In a step 512, upon detection of chemically detected sample (in step 510), one or more actions may be performed to prevent the chemically contaminated sample from being inserted into the inspection chamber. For example, the one or more processors 114 of the controller 112 may alert a user (or host tool) of the presence of the chemically contaminated sample 104 (or “wet” sample). In one non-limiting instance, inspection of the sample 104 may be paused until the sample 104 is dry. In another non-limiting instance, the chemically contaminated sample 104 may be rejected. As such, users may be able to use such data to adjust their process tools and/or cleaning process.

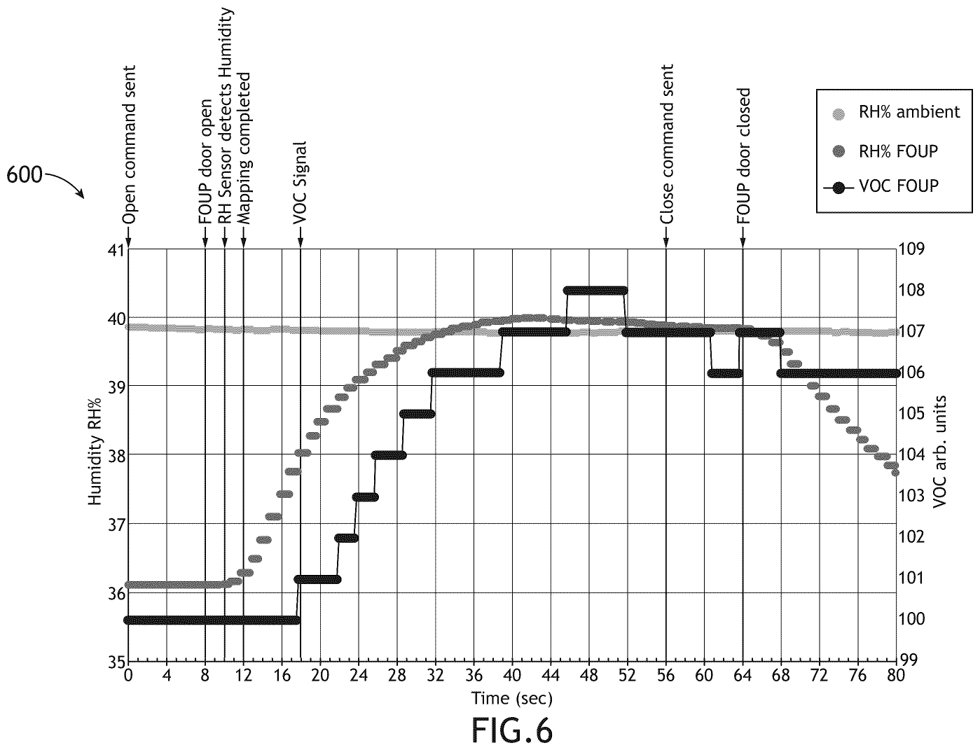

The detection scheme of the present disclosure may provide a number of advantages/benefits. For example, it is contemplated herein that the detection of chemically contaminated samples using the system 100 does not affect throughput due to the fast detection scheme of the present disclosure. FIG. 6 is a plot 600 depicting humidity and volatile organic content signals over time, in accordance with one or more embodiments of the present disclosure. In a non-limiting example, as shown in FIG. 6, the FOUP door opens at approximately 8 seconds and humidity is detected by the environmental sensor device 106 at approximately 10 seconds (i.e., within approximately 2-3 seconds). Mapping by the sample mapper is not completed until approximately 12 seconds, as such, the process already accounts for the time needed to detect humidity and thus, does not affect throughput. It is noted herein that FIG. 6 is provided merely for illustrative purposes and shall not be construed as limiting the scope of the present disclosure. The steps and respective timing may be dependent on specifications of the load handler, or other components of the system.

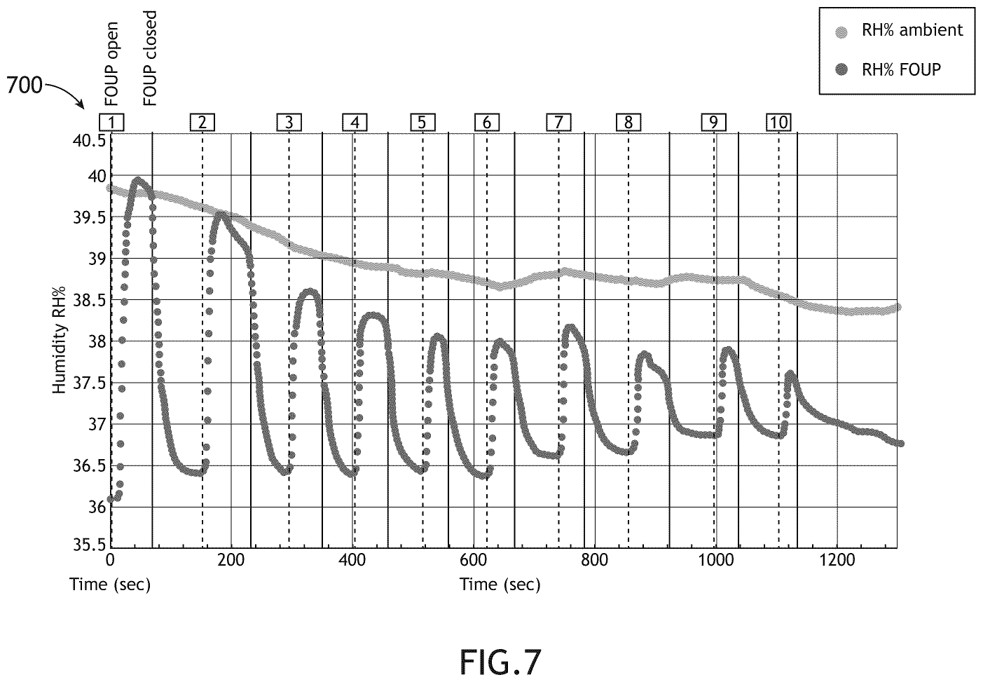

Further, it is contemplated herein that the detection scheme of the present disclosure is robust. FIG. 7 is a plot 700 depicting humidity signals over time as the sample transport device door is open and closed, in accordance with one or more embodiments of the present disclosure. For example, as shown in FIG. 7, the humidity signal decreases overtime as the FOUP door is opened and closed, however, the environmental sensor device 106 is still able to detect a humidity signal even after approximately 20 minutes and 10 open/close cycles.

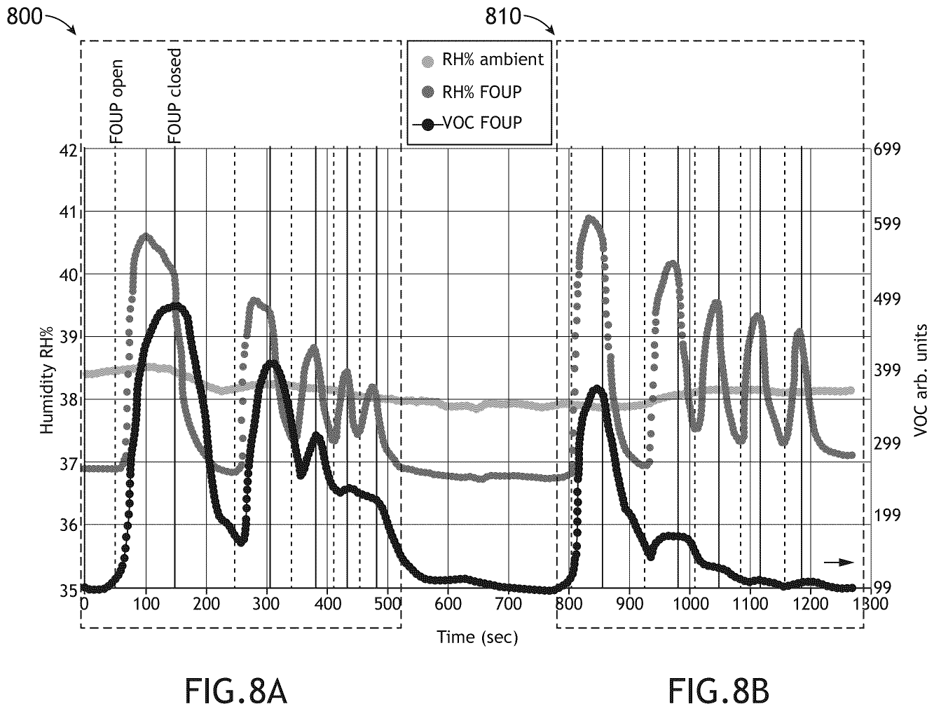

As previously noted herein, the detection scheme of the present disclosure is able to detect chemical contamination caused by both water (i.e., humidity) and organic compounds. In this regard, where the sample is sprayed with an organic compound such as, but not limited to, isopropyl alcohol, acetone, or the like, after the cleaning process, the detection scheme may be able to detect the presence of the organic compounds. For example, FIG. 8A is a plot 800 depicting humidity and volatile organic content signals over time for water and isopropyl alcohol, in accordance with one or more embodiments of the present disclosure. As shown, a humidity signal caused by the water (i.e., deionized (DI) water) and a VOC signal caused by the isopropyl alcohol (IPA) is simultaneously detected by the environmental sensor device 106. By way of another example, FIG. 8B is a plot 810 depicting humidity and volatile organic content signals over time for water and acetone, in accordance with one or more embodiments of the present disclosure. As shown, a humidity signal caused by the water (i.e., deionized (DI) water) and a VOC signal caused by the acetone is simultaneously detected by the environmental sensor device 106.

Referring to FIG. 1, the one or more processors 114 of a controller 112 may include any processing element known in the art. In this sense, the one or more processors 114 may include any microprocessor-type device configured to execute algorithms and/or instructions. In embodiments, the one or more processors 114 may consist of a desktop computer, mainframe computer system, workstation, image computer, parallel processor, or any other computer system (e.g., networked computer) configured to execute a program configured to operate the mini-environment control system 100, as described throughout the present disclosure. It is further recognized that the term “processor” may be broadly defined to encompass any device having one or more processing elements, which execute program instructions from a non-transitory memory medium 116.

The memory medium 116 may include any storage medium known in the art suitable for storing program instructions executable by the associated one or more processors 114. For example, the memory medium 116 may include a non-transitory memory medium. By way of another example, the memory medium 116 may include, but is not limited to, a read-only memory, a random-access memory, a magnetic or optical memory device (e.g., disk), a magnetic tape, a solid-state drive and the like. It is further noted that memory medium 116 may be housed in a common controller housing with the one or more processors 114. In embodiments, the memory medium 116 may be located remotely with respect to the physical location of the one or more processors 114 and controller 112. For instance, the one or more processors 114 of controller 112 may access a remote memory (e.g., server), accessible through a network (e.g., internet, intranet and the like). Therefore, the above description should not be interpreted as a limitation on the present disclosure but merely an illustration.

It is contemplated that each of the embodiments of the method described above may include any other step(s) of any other method(s) described herein. In addition, each of the embodiments of the method described above may be performed by any of the systems described herein.

One skilled in the art will recognize that the herein described components operations, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components, operations, devices, and objects should not be taken as limiting.

As used herein, directional terms such as “top,” “bottom,” “over,” “under,” “upper,” “upward,” “lower,” “down,” and “downward” are intended to provide relative positions for purposes of description, and are not intended to designate an absolute frame of reference. Various modifications to the described embodiments will be apparent to those with skill in the art, and the general principles defined herein may be applied to other embodiments.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected,” or “coupled,” to each other to achieve the desired functionality, and any two components capable of being so associated can also be viewed as being “couplable,” to each other to achieve the desired functionality. Specific examples of couplable include but are not limited to physically mateable and/or physically interacting components and/or wirelessly interactable and/or wirelessly interacting components and/or logically interacting and/or logically interactable components.

Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” and the like). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, and the like” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, and the like). In those instances where a convention analogous to “at least one of A, B, or C, and the like” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, and the like). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed is:

1. A sample contamination detection assembly comprising:
an environmental sensor device, wherein the environmental sensor device is configured to simultaneously measure at least two or more of humidity, temperature, or volatile organic content of one or more samples to determine chemical contamination of the one or more samples; and
a funnel device, wherein the funnel device comprises:
a plurality of sidewalls that define two or more internal cavities, wherein the two or more internal cavities include at least an inlet cavity configured to receive inlet air and an exhaust cavity configured to receive exhaust air, wherein at least one sidewall of the plurality of sidewalls includes an inlet opening connected to the inlet cavity and a first exhaust opening connected to the exhaust cavity, wherein the environmental sensor device is arranged proximate to the inlet opening and the first exhaust opening on the at least one sidewall of the funnel device, wherein at least one additional sidewall of the plurality of sidewalls includes an additional exhaust opening;
a lip configured to direct handler air away from the environmental sensor device; and
a slit arranged on at least one sidewall of the plurality of sidewalls, wherein the slit is connected to the inlet cavity, wherein the slit is configured to direct air from the one or more samples to the environmental sensor device via the inlet cavity and the inlet opening of the funnel device.

2. The sample contamination detection assembly of claim 1, wherein the plurality of sidewalls of the funnel device includes at least a top sidewall, a bottom sidewall, a front sidewall, a rear sidewall, and two or more side sidewalls.

3. The sample contamination detection assembly of claim 2, wherein the slit is arranged on the bottom sidewall of the funnel device and the environmental sensor device is arranged proximate to the top sidewall of the funnel device.

4. The sample contamination detection assembly of claim 1, wherein the environmental sensor device includes a fan configured to direct the exhaust air to the exhaust cavity via the first exhaust opening.

5. The sample contamination detection assembly of claim 4, further comprising:
an exhaust tube configured to couple to the additional exhaust opening on the at least one additional sidewall of the funnel device, wherein the exhaust tube is configured to direct the exhaust air in the exhaust cavity away from the one or more samples.

6. The sample contamination detection assembly of claim 1, further comprising:
one or more reference sensor devices.

7. The sample contamination detection assembly of claim 6, wherein the one or more reference sensor devices include an ambient humidity sensor.

8. A system for detecting chemical contamination comprising:
a sample contamination detection assembly, the sample contamination detection assembly comprising:
an environmental sensor device, wherein the environmental sensor device is configured to simultaneously measure at least two or more of humidity, temperature, or volatile organic content (VOC) of one or more samples; and
a funnel device, wherein the funnel device comprises:
a plurality of sidewalls that define two or more internal cavities, wherein the two or more internal cavities include at least an inlet cavity configured to receive inlet air and an exhaust cavity configured to receive exhaust air, wherein at least one sidewall of the plurality of sidewalls includes an inlet opening connected to the inlet cavity and a first exhaust opening connected to the exhaust cavity, wherein the environmental sensor device is arranged proximate to the inlet opening and the first exhaust opening on the at least one sidewall of the funnel device, wherein at least one additional sidewall of the plurality of sidewalls includes an additional exhaust opening;
a lip configured to direct handler air away from the environmental sensor device; and
a slit arranged on at least one sidewall of the plurality of sidewalls, wherein the slit is connected to the inlet cavity, wherein the slit is configured to direct air from the one or more samples to the environmental sensor device via the inlet cavity and the inlet opening of the funnel device; and
a controller communicatively coupled to the sample contamination detection assembly, the controller configured to execute program instructions causing one or more processors to:
receive one or more reference signals from one or more reference sensor devices;
receive one or more sample contamination signals from the environmental sensor device, wherein the one or more sample contamination signals include the simultaneously measured at least two or more of humidity, temperature, or volatile organic content of the one or more samples;
determine chemical contamination of the one or more samples by comparing the received one or more references signals, the one or more sample contamination signals, and one or more predetermined thresholds; and
upon determination of chemical contamination of the one or more samples, prevent the one or more samples from being loaded into an inspection chamber of an inspection tool.

9. The system of claim 8, wherein the controller is further configured to:
upon determination of chemical contamination of the one or more samples, generate one or more alerts; and
provide the generated one or more alerts to a user to notify the user of the determination of chemical contamination of the one or more samples.

10. The system of claim 8, wherein the one or more predetermined thresholds are humidity levels between 5% and 10%.

11. The system of claim 8, wherein the one or more predetermined thresholds are VOC levels between 5% and 10%.

12. The system of claim 8, wherein the controller is further configured to:
receive one or more sample mapper signals from a sample mapper, the one or more sample mapper signals including sample position data and sample mapper position data.

13. The system of claim 8, wherein the plurality of sidewalls of the funnel device include at least a top sidewall, a bottom sidewall, a front sidewall, a rear sidewall, and two or more side sidewalls.

14. The system of claim 13, wherein the slit is arranged on the bottom sidewall of the funnel device and the environmental sensor device is arranged proximate to the top sidewall of the funnel device.

15. The system of claim 14, wherein the environmental sensor device includes a fan configured to direct the exhaust air to the exhaust cavity via the first exhaust opening.

16. The system of claim 15, further comprising:
an exhaust tube configured to couple to the additional exhaust opening on the at least one additional sidewall of the funnel device, wherein the exhaust tube is configured to direct the exhaust air in the exhaust cavity away from the one or more samples.

17. A system comprising:
a load port device, wherein the load port device is configured to receive one or more samples from a portion of a sample transport device;
an automated handling sub-system, wherein the automated handling sub-system is configured to extract the one or more samples from the sample transport device, and
a sample contamination detection assembly, the sample contamination detection assembly arranged proximate to a load port door of the load port device; the sample contamination detection assembly comprising:
an environmental sensor device, wherein the environmental sensor device is configured to simultaneously measure at least two or more of humidity, temperature, or volatile organic content of the one or more samples to determine chemical contamination of the one or more samples; and
a funnel device, wherein the funnel device comprises:
a plurality of sidewalls that define two or more internal cavities, wherein the two or more internal cavities include at least an inlet cavity configured to receive inlet air and an exhaust cavity configured to receive exhaust air, wherein at least one sidewall of the plurality of sidewalls includes an inlet opening connected to the inlet cavity and a first exhaust opening connected to the exhaust cavity, wherein the environmental sensor device is arranged proximate to the inlet opening and the first exhaust opening on the at least one sidewall of the funnel device, wherein at least one additional sidewall of the plurality of sidewalls includes an additional exhaust opening;
a lip configured to direct air from the automated handling sub-system away from the environmental sensor device; and
a slit arranged on at least one sidewall of the plurality of sidewalls, wherein the slit is connected to the inlet cavity, wherein the slit is configured to direct air from the one or more samples to the environmental sensor device via the inlet cavity and the inlet opening of the funnel device.

18. The system of claim 17, further comprising:
a controller communicatively coupled to the sample contamination detection assembly.

19. The system of claim 18, wherein the controller is configured to execute program instructions causing one or more processors to:
receive one or more reference signals from one or more reference sensor devices;
receive one or more sample contamination signals from the environmental sensor device, wherein the one or more sample contamination signals include the simultaneously measured at least two or more of humidity, temperature, or volatile organic content of the one or more samples;
determine chemical contamination of the one or more samples by comparing the received one or more references signals, the one or more sample contamination signals, and one or more predetermined thresholds; and
upon determination of chemical contamination of the one or more samples, prevent the one or more samples from being loaded into an inspection chamber of an inspection tool via the automated handling sub-system.

20. A method comprising:
opening a lid of a sample transport device using a load port door;
receiving one or more reference signals from one or more reference sensor devices;
receiving one or more sample contamination signals from an environmental sensor device, wherein the environmental sensor device is configured to simultaneously measure at least two or more of humidity, temperature, or volatile organic content of one or more samples;
determining chemical contamination of the one or more samples by comparing the received one or more references signals, the one or more sample contamination signals, and one or more predetermined thresholds; and
upon determination of chemical contamination of the one or more samples, preventing the one or more samples from being loaded into an inspection chamber of an inspection tool.

21. The method of claim 20, wherein further comprising:
upon determination of chemical contamination of the one or more samples, generate one or more alerts; and
providing the generated one or more alerts to a user to notify the user of the determination of chemical contamination of the one or more samples.

22. The method of claim 20, wherein the one or more predetermined thresholds are humidity levels between 5% and 10%.

23. The method of claim 20, wherein the one or more predetermined thresholds are VOC levels between 5% and 10%.

24. The method of claim 20, further comprising:
loading the sample transport device with the one or more samples.

25. The method of claim 20, further comprising:
receiving one or more sample mapper signals from a sample mapper, the one or more sample mapper signals including sample position data and sample mapper position data.
